(19) United States
(12) Patent Application Publication
(10) Pub. No.: US 2013/0289488 A1
(43) Pub. Date: Oct. 31, 2013

(54) Dedicated Cartridge and Holder

(71) Applicants: Ralf Riess, Frankfurt am Main (DE); Stefan Trinkle, Frankfurt am Main (DE); James Senior, Warwickshire (GB); James Alexander Davies, Warwickshire (GB); Andrew Gordon Wallace, Gloucestershire (GB)

(72) Inventors: Ralf Riess, Frankfurt am Main (DE); Stefan Trinkle, Frankfurt am Main (DE); James Senior, Warwickshire (GB); James Alexander Davies, Warwickshire (GB); Andrew Gordon Wallace, Gloucestershire (GB)

(73) Assignee: SANOFI-AVENTIS DEUTSCHLAND GMBH, Frankfurt am Main (DE)

(21) Appl. No.: 13/997,965

(22) PCT Filed: Dec. 20, 2011
(86) PCT No.: PCT/EP2011/073818

§ 371 Date: Jun. 25, 2013

Related U.S. Application Data
(60) Provisional application No. 61/427,362, filed on Dec. 27, 2010.

(30) Foreign Application Priority Data
Feb. 4, 2011 (EP) 11153434.3

(51) Int. Cl. A61M 5/31

(52) U.S. Cl. CPC A61M 5/31
USPC 604/189

(57) ABSTRACT

A cartridge assembly for use with a drug delivery device comprises:

a cartridge holder defining an aperture therethrough, with a blocking feature and at least one holder coding feature; and
a cartridge with at least one cartridge coding feature;
wherein when the cartridge is inserted into the cartridge holder, the cartridge coding feature mates with the holder coding feature, moving the blocking feature or allowing the blocking feature to move.

### CROSS REFERENCE TO RELATED APPLICATIONS

The present application is a U.S. National Phase Application pursuant to 35 U.S.C. §371 of International Application No. PCT/EP2011/073818 filed Dec. 22, 2011, which claims priority to U.S. Provisional Patent Application No. 61/427,362 filed Dec. 27, 2010 and European Patent Application No. 11153434.3 filed Feb. 4, 2011. The entire disclosure contents of these applications are herewith incorporated by reference into the present application.

### FIELD OF INVENTION

The present disclosure is generally directed to reservoirs, particularly reservoirs containing a medicament. More particularly, the present disclosure is generally directed to a coding mechanism for use with a reservoir and a reservoir holder so as to ensure the reservoir and reservoir holders are only used with the drug for which they are intended. As just one example, such medicament reservoirs may comprise an ampoule, a cartridge, a vial, or a pouch, and may be used with a medical delivery device. Exemplary medical delivery devices include, but are not limited to syringes, pen type injection syringes, pumps, inhalers, or other similar injection or infusing devices that require at least one reservoir containing at least one medicament.

### BACKGROUND

Medicament reservoirs such as ampoules, cartridges, or vials are generally known. Such reservoirs are especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge holder. After the cartridge has been correctly loaded, the user may then be called upon to select a dose of medicament. Multiple doses may be dosed from the cartridge. Where the drug delivery device comprises a reusable device, once the cartridge is empty, the cartridge holder is disconnected from the drug delivery device and the empty cartridge is removed and replaced with a new cartridge. Most suppliers of such cartridges recommend that the user dispose of the empty cartridges properly. Where the drug delivery device comprises a disposable device, once the cartridge is empty, the user is recommended to dispose of the entire device.

Such known self administration systems requiring the removal and reloading of empty cartridges have certain limitations. For example, in certain generally known systems, a user simply loads a new cartridge into the delivery system without the drug delivery device or without the cartridge having any mechanism of preventing removal and subsequent cross use of an incorrect cartridge. Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short acting insulin in lieu of a long insulin could result in injury or even death.

Some drug delivery devices or systems may use a color coding scheme to assist a user or care giver in selecting the correct cartridge to be used with a drug delivery device. However, such color coding schemes pose challenges to certain users, especially those users suffering from poor eyesight or color blindness: a situation that can be quite prevalent in patients suffering from diabetes.

Another concern that may arise with such disposable cartridges is that these cartridges are manufactured in essentially standard sizes and must comply with certain recognized local and international standards. Consequently, such cartridges are typically supplied in standard sized cartridges (e.g., 3 ml cartridges). Therefore, there may be a variety of cartridges supplied by a number of different suppliers and containing different medicament but they may fit a single drug delivery device. As just one example, a first cartridge containing a first medicament from a first supplier may fit a medical delivery device provided by a second supplier. As such, a user might be able to load and then dispense an incorrect medicament (such as a rapid or basal type of insulin) into a drug delivery device without being aware that the medical delivery device was perhaps neither designed nor intended to be used with such a cartridge.

As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

There is, therefore, a general need to physically dedicate or mechanically code a cartridge and cartridge holder to its drug type and design an injection device that accepts or works with the dedication or coded features provided on or with the cartridge so as to prevent unwanted cartridge cross use. Similarly, there is also a general need for a dedicated cartridge and cartridge holder that allows the medical delivery device to be used with an authorized cartridge containing a specific medicament while also preventing undesired cartridge cross use.

There is also a general need to provide a dedicated cartridge and cartridge holder that is difficult to tamper with so that the cartridge may not be compromised in that the cartridge can be used with an unauthorized drug or drug delivery device. Because such cartridges may be difficult to tamper with, they may also reduce the risk of counterfeiting: i.e., making it more difficult for counterfeiters to provide unregulated counterfeit medicament carrying products.

It is an aim to provide an improved cartridge assembly with a cartridge and a cartridge holder for use with a drug delivery system.

### SUMMARY

Medicament reservoirs such as ampoules, cartridges, or vials are generally known. Such reservoirs are especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge holder. After the cartridge has been correctly loaded, the user may then be called upon to select a dose of medicament. Multiple doses may be dosed from the cartridge. Where the drug delivery device comprises a reusable device, once the cartridge is empty, the cartridge holder is disconnected from the drug delivery device and the empty cartridge is removed and replaced with a new cartridge. Most suppliers of such cartridges recommend that the user dispose of the empty cartridges properly. Where the drug delivery device comprises a disposable device, once the cartridge is empty, the user is recommended to dispose of the entire device.

Such known self administration systems requiring the removal and reloading of empty cartridges have certain limitations. For example, in certain generally known systems, a user simply loads a new cartridge into the delivery system without the drug delivery device or without the cartridge having any mechanism of preventing removal and subsequent cross use of an incorrect cartridge. Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short acting insulin in lieu of a long insulin could result in injury or even death.

Some drug delivery devices or systems may use a color coding scheme to assist a user or care giver in selecting the correct cartridge to be used with a drug delivery device. However, such color coding schemes pose challenges to certain users, especially those users suffering from poor eyesight or color blindness: a situation that can be quite prevalent in patients suffering from diabetes.

Another concern that may arise with such disposable cartridges is that these cartridges are manufactured in essentially standard sizes and must comply with certain recognized local and international standards. Consequently, such cartridges are typically supplied in standard sized cartridges (e.g., 3 ml cartridges). Therefore, there may be a variety of cartridges supplied by a number of different suppliers and containing different medicament but they may fit a single drug delivery device. As just one example, a first cartridge containing a first medicament from a first supplier may fit a medical delivery device provided by a second supplier. As such, a user might be able to load and then dispense an incorrect medicament (such as a rapid or basal type of insulin) into a drug delivery device without being aware that the medical delivery device was perhaps neither designed nor intended to be used with such a cartridge.

As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

There is, therefore, a general need to physically dedicate or mechanically code a cartridge and cartridge holder to its drug type and design an injection device that accepts or works with the dedication or coded features provided on or with the cartridge so as to prevent unwanted cartridge cross use. Similarly, there is also a general need for a dedicated cartridge and cartridge holder that allows the medical delivery device to be used with an authorized cartridge containing a specific medicament while also preventing undesired cartridge cross use.

There is also a general need to provide a dedicated cartridge and cartridge holder that is difficult to tamper with so that the cartridge may not be compromised in that the cartridge can be used with an unauthorized drug or drug delivery device. Because such cartridges may be difficult to tamper with, they may also reduce the risk of counterfeiting: i.e., making it more difficult for counterfeiters to provide unregulated counterfeit medicament carrying products.

It is an aim to provide an improved cartridge assembly with a cartridge and a cartridge holder for use with a drug delivery system.

### BRIEF DESCRIPTION OF THE DRAWINGS

Exemplary embodiments are described herein with reference to the drawings, in which:

FIG. 1 illustrates an exemplary pen type drug delivery device;

FIG. 2 illustrates a cartridge that may be loaded into a cartridge holder of the pen type drug delivery device illustrated in FIG. 1;

FIG. 3 illustrates a first arrangement of an exemplary cartridge and cartridge holder that may be used with a pen type drug delivery device, such as the drug delivery device illustrated in FIG. 1;

FIG. 4 illustrates an enlarged, partial view of the cartridge and cartridge holder of FIG. 3;

FIG. 5 illustrates another arrangement view of an exemplary cartridge and cartridge holder that may be used with a pen type drug delivery device, such as the drug delivery device illustrated in FIG. 1;

FIG. 6 illustrates another arrangement of an exemplary cartridge and cartridge holder that may be used with a pen type drug delivery device, such as the drug delivery device illustrated in FIG. 1;

FIG. 7 illustrates an enlarged, partial view of the cartridge and cartridge holder of FIG. 6;

FIG. 8 illustrates an enlarged, partial view of the cartridge and cartridge holder of FIG. 6, where the cartridge is fully inserted within the shuttle of the cartridge holder;

FIG. 9 illustrates the fully inserted cartridge and cartridge holder of FIG. 6;

FIG. 10A illustrates yet another alternative arrangement of an exemplary cartridge and cartridge holder that may be used with a pen type drug delivery device, such as the drug delivery device illustrated in FIG. 1;

FIG. 10B illustrates the cartridge and cartridge holder of FIG. 10A in a second position;

FIG. 10C illustrates the cartridge and cartridge holder of FIG. 10A in a third position; and

FIG. 10D illustrates the cartridge and cartridge holder of FIG. 10A in a fourth position.

### DETAILED DESCRIPTION

The terms “medicament” and “drug”, as used herein, preferably mean a pharmaceutical formulation containing at least one pharmaceutically active compound,
wherein in one embodiment the pharmaceutically active compound has a molecular weight up to 1500 Da and/or is a peptide, a protein, a polysaccharide, a vaccine, a DNA, a RNA, an enzyme, an antibody, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
wherein the group -Lys6-NH2 may be bound to the C-terminus of the Exendin-4 derivative;
or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5-des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

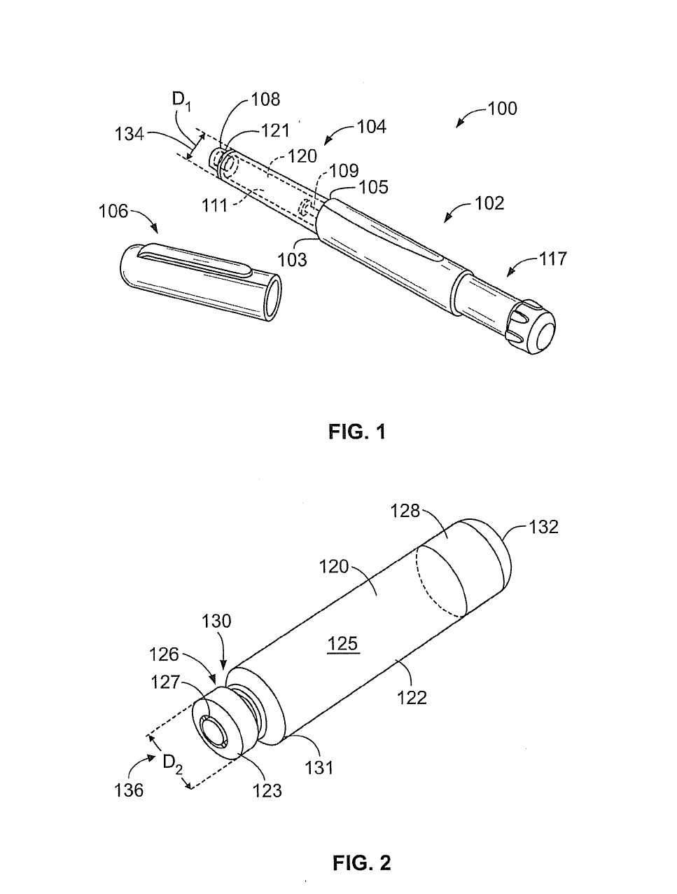

Referring to FIG. 1, there is shown a drug delivery device 100 in the form of a pen type syringe. This drug delivery device 100 comprises a dose setting mechanism 102, a cartridge holder 104, and a removable cap 106. A proximal end 105 of the cartridge holder 104 and a distal end 103 of the dose setting mechanism 102 are removably secured together. The dose setting mechanism 102 comprises a piston rod 109, such as a threaded piston rod that rotates when a dose is injected.

To inject a previously set dose, a double ended needle assembly (not shown) is attached to a distal end 108 of the cartridge holder 104. Preferably, the distal end 108 of the cartridge holder 104 comprises a thread 121 (or other suitable connecting mechanism such as a snap lock, snap fit, form fit, or bayonet lock mechanism) so that the needle assembly may be removably attached to the distal end 108 of the cartridge holder 104. When the drug delivery device 100 is not in use, the removable cap 106 can be releasably retained over the cartridge holder 104.

An inner cartridge cavity 111 defined by the cartridge holder 104 is dimensioned and configured to securely receive and retain the cartridge 120. FIG. 2 illustrates a perspective view of the cartridge 120 that may be used with the drug delivery device 100 illustrated in FIG. 1. The cartridge 120 includes a generally tubular barrel 122 extending from a distal end 130 to a proximal end 132. The distal end 130 is defined by an inwardly converging shoulder 131.

At the distal end 130, the cartridge 120 includes a smaller diameter neck 126 and this neck 126 projects distally from the shoulder 131 of the barrel 122. Preferably, this smaller diameter neck 126 is provided with a large diameter annular bead (not shown) and this bead extends circumferentially thereabout at the extreme distal end of the neck 126. A pierceable seal 127 or septum is securely mounted across the open distal end 130 defined by the neck 126. The seal 127 may be held in place by a metallic sleeve or ferrule 123. This ferrule 123 may be crimped around the circumferential bead at the distal end of the neck 126. The diameter of ferrule 123 is shown by D₂ 136. The medicament 125 is pre-filled into the cartridge 120 and is retained within the cartridge 120, in part, by the pierceable seal 127, the metallic sleeve or ferrule 123, and the stopper 128. The stopper 128 is in sliding fluid-tight engagement with the inner tubular wall of the barrel 122. Axially directed forces acting upon the stopper 128 during dose injection or dose administration urges the medicament 125 from the cartridge 120 though a double ended needle mounted onto the distal end 130 of the cartridge holder 104 and into the injection site. Such axial forces may be provided by the piston rod 109.

A portion of the cartridge holder 104 defining the cartridge holder cavity 111 is of substantially uniform diameter represented in FIG. 1 by D₁ 134. This diameter D₁ is preferably slightly greater than the diameter D₂ of the cartridge 120. The interior of the cartridge holder 104 includes an inwardly-extending annual portion or stop that is dimensioned to prevent the cartridge 120 from moving within the cartridge holder 104. In this manner, when the cartridge 120 is loaded into the cavity 111 of the cartridge holder 104 and the cartridge holder 104 is then connected to the dose setting mechanism 102, the cartridge 120 will be securely held within the cartridge cavity 111. More particularly, the neck 126 and ferrule 123 of the cartridge 120 are inserted in a proximal to distal direction into the open proximal end 105 of the cartridge holder 104 with the ferrule 123 eventually passing entirely into the cartridge holder 104. With the cartridge holder 104 removably coupled to the dose setting mechanism 102, the proximal end 132 of the cartridge 120 will typically abut a stop provided by the dose setting mechanism 102.

A number of doses of a medicament 125 may be dispensed from the cartridge 120. Preferably, the cartridge 120 contains a type of medicament 125 that is administered often, such as one or more times a day. One such medicament 125 is insulin. The stopper 128 is retained in a first end or proximal end 132 of the cartridge 120 and receives an axial force created by the piston rod 109 of the dose setting mechanism 102.

The dose setting mechanism 102 comprises a dose setter 117 at the proximal end of the dose setting mechanism 102. In one preferred arrangement, the dose setter 117 is rotated to set a dose. To administer this set dose, the user attaches the needle assembly comprising a double ended needle on the distal end 108 of the cartridge holder 104. In this manner, the needle assembly pierces the seal 127 of the cartridge 120 and is therefore in liquid communication with the medicament 125. The user pushes on the dose setter 117 to inject the set dose. The same dose setting and dose administration procedure is followed until the medicament 125 in the cartridge 120 is expended, at which time a new cartridge 120 may be loaded in the drug delivery device 100. To exchange an empty cartridge 120, the user is called upon to remove the cartridge holder 104 from the dose setting mechanism 102.

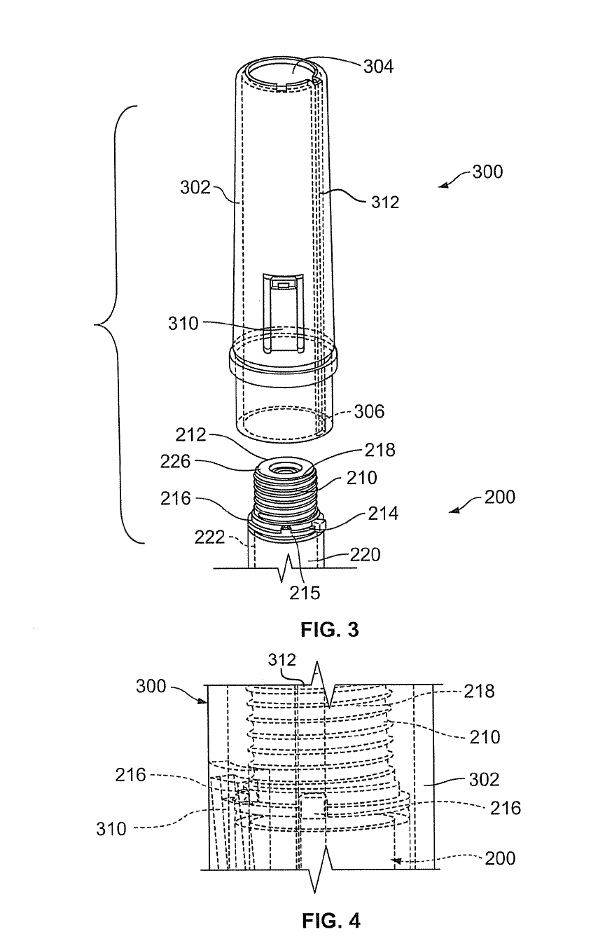

FIG. 3 is a first exemplary arrangement of a cartridge 200 for use with a cartridge holder 300. Cartridge 200 and cartridge holder 300 are for use with a pen type drug delivery device, such as drug delivery device 100 illustrated in FIGS. 1 and 2.

Cartridge 200 comprises a main body 220 with an exterior surface 222, a distal end 226, and a proximal end (not shown). Cartridge 200 is fitted with a cap 210. Cap 210 may be a separate piece that is attached to cartridge 200, or may be molded as part of cartridge 200. Cap 210 comprises a distal end 212 and a proximal end 214, and includes a threaded portion 218 as well as coding and alignment features 216 positioned along the periphery of proximal end 214. At proximal end 214 is a shoulder 215, which extends radially from the main body of adaptor cap 210. Coding and alignment features 216 may comprise a number of features along shoulder 215. For example, coding and alignment features 216 may, as just one example, comprise a series of indents and protrusions on shoulder 215.

Cartridge holder 300 comprises a main body 302, a distal end 304, and a proximal end 306. Cartridge holder 300 also comprises at least one clip mechanism 310 and a groove 312.

The coding and alignment features 216 on cap 210 serve as a coding mechanism. More specifically, as cartridge 200 is axially inserted into the cartridge holder 300 from the proximal end 304 of the cartridge holder 300, coding and alignment features 216 engage with clip mechanism 310 and groove 312 of holder 300.

A cartridge 200 can thus have a cap 210 that allows the cartridge 200 to be designated to fit only within its proper cartridge holder 300. When a cartridge 200 is properly paired with its corresponding cartridge holder 300, the coding and alignment features 216 on cap 210 engage clip 310 when cartridge 200 is inserted into cartridge holder 300. Once clip 310 is properly engaged by a coding feature 216, the clip 310 is deflected radially outwards from cartridge holder 300, allowing cap 210 to pass past clip 310 and the cartridge 200 to thus be fully inserted into cartridge holder 300.

If coding and alignment features 216 are either not present on a cartridge 200 or are located in the wrong location relative to the clip mechanism 310, such that clip mechanism 310 is not aligned with the coding and alignment features features 216, the clip mechanism 310 will not be deflected. Thus, upon cartridge insertion, the clip mechanism 310 will interfere with the shoulder 215 of the cap 210, preventing the cartridge holder 300 from proceeding over cartridge 200 beyond the clip 310. That is, the cartridge travel will be restricted, and cartridge 200 will not be able to continue to be inserted within cartridge holder 300.

FIG. 4 illustrates an enlarged, partial view of the cartridge 200 inserted within cartridge holder 300 of FIG. 3. In this enlarged view, it can be seen that coding and alignment features 216 may comprise a block that protrudes from the cartridge 200 that slides within groove 312 in the cartridge holder 300. FIG. 4 also shows another coding and alignment feature 216 engaging clip mechanism 310, pushing it radially outward.

In an alternative configuration, the clip mechanism 310 could be engaged by means other than coding features 216 on a cap 210, for example by features on a coded label, coded ferrule, or even features formed in the glass cartridge 200 itself.

Threaded portion 218 on cap 210 allows for the attachment of a needle. The cartridge 200 may be configured such that without the cap 210, it is not possible to attach a needle to the cartridge 200.

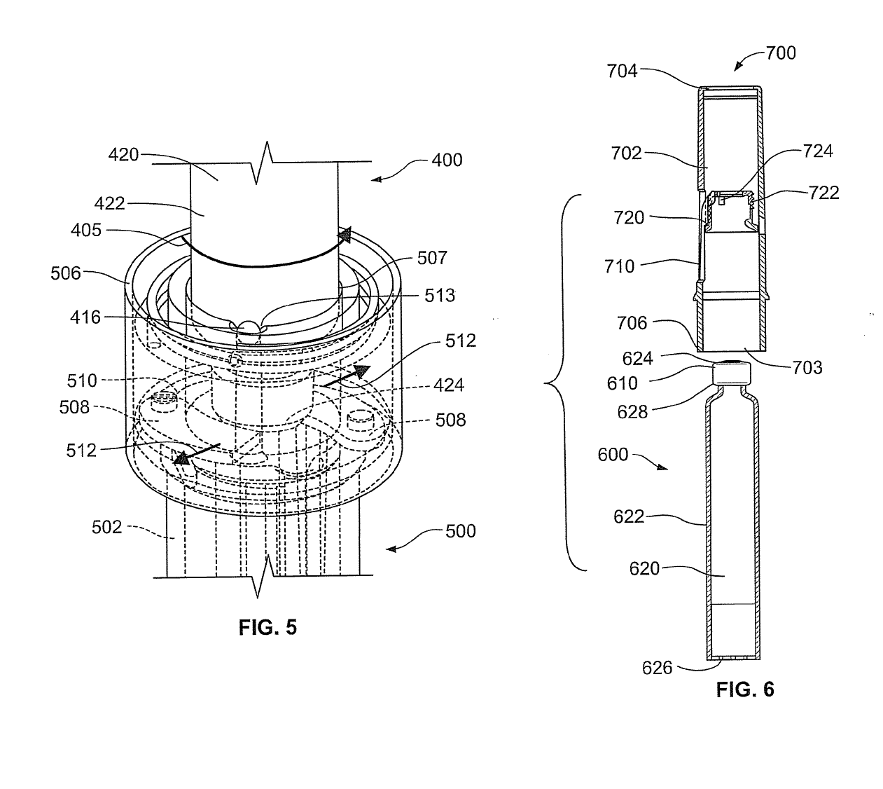

FIG. 5 illustrates another exemplary cartridge 400 for use with a cartridge holder 500. Cartridge 400 comprises a main body 420 with an exterior surface 422, a distal end 424, and a proximal end (not shown). Cartridge 400 comprises coding features 416. Coding features 416 may comprise spherical orbs, as shown in FIG. 5. These coding features 416 may be present on a ferrule, label, cap, or may be formed directly in or on the glass cartridge 400.

Cartridge holder 500 comprises a main body 502, a distal end (not shown), and a proximal end 506 with an aperture 507. Cartridge holder 500 also comprises a gate or blocking mechanism, which in FIG. 5 is shown as two petals 508, and an inner rotating sleeve 510. Inner rotating sleeve 510 is preferably mechanically linked to petals 508. Inner rotating sleeve 510 may comprise an indent 513 or other feature that corresponds with the coding feature 416 on the appropriate cartridge 400.

In the initial or rest position, the blocking mechanism prevents the entry of any cartridge 400 into cartridge holder 500. When the correct cartridge 400 is inserted into the aperture 507, coding features 416 first engage the indent 513 in rotating sleeve 510. Once coding features 416 are engaged with rotating sleeve 510, a user can manually rotate cartridge 400 in the direction shown by arrow 405, which will in turn rotate the attached inner rotating sleeve 510. The rotation of inner rotating sleeve 510 causes the linked petals 508 to retract, moving away from blocking the path of insertion of cartridge 400. That is, the linked petals 508 move toward the inner walls of cartridge holder 500 (in the direction shown by arrows 512), away from aperture 507.

In this manner, coding features 416 can provide a coding feature to the cartridge 400 to allow for assembly with a corresponding cartridge holder 500. If an incorrect cartridge insertion is attempted, the cartridge travel will be restricted and the cartridge 400 will not be able to engage with the cartridge holder 500, preventing the cartridge 400 from being fully inserted into cartridge holder 500. Thus, no drug can be dispensed.

FIG. 6 illustrates another arrangement of a cartridge 600, for use with a cartridge holder 700. Cartridge 600 comprises a main body 620 with an exterior surface 622, a distal end 624, and a proximal end 626. At distal end 624 is a ferrule 628. Cartridge 600 is also fitted with at least one coding feature 610 on the ferrule 628. Ferrule 628 may be molded as part of cartridge 600.

Cartridge holder 700 comprises a main body 702 defining an aperture 703, a distal end 704, and a proximal end 706. Cartridge holder 700 also comprises at least one clip 710, which is connected to a shuttle 720. Clip 710 is on the exterior of cartridge holder 700, and extends through the interior of cartridge holder 700 so as to attach to shuttle 720. Shuttle 720 may be a cap with a needle thread 722 on its exterior surface, and is preferably sized and shaped to accept the ferrule 628 of cartridge 600. The interior surface of shuttle 720 may comprise at least one coding feature 724.

Initially, the shuttle 720 is retained within cartridge holder 700 by clip 710. Clip 710 will only release shuttle 720 from its initial position if it is deflected away from the shuttle 720, which occurs upon contact with the proper ferrule 610.

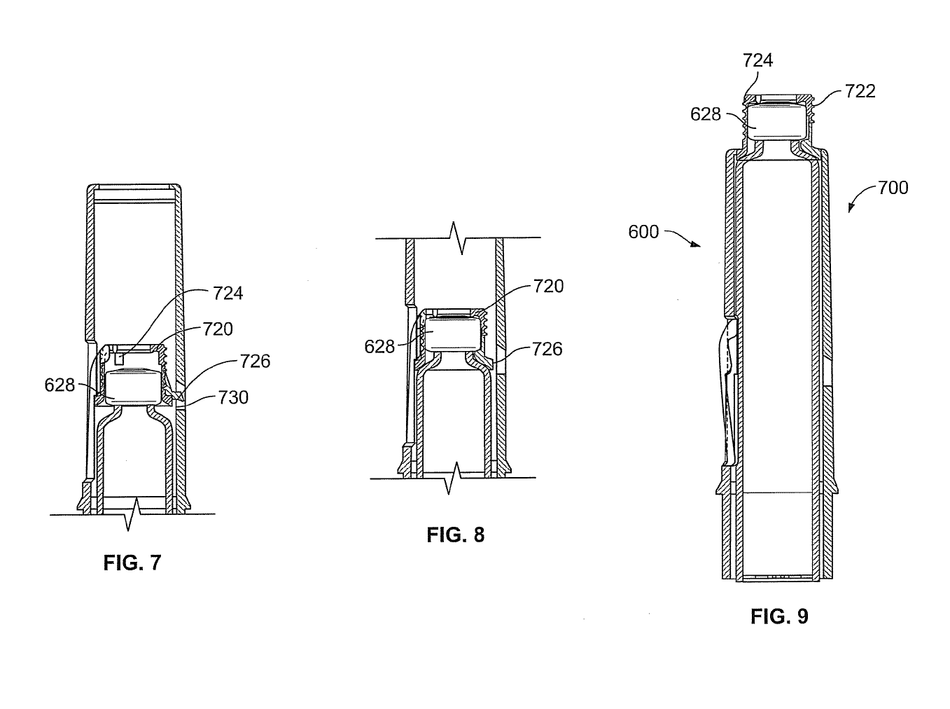

FIGS. 7 and 8 are enlarged, partial views of the insertion of cartridge 600 into cartridge holder 700, and demonstrate the interaction of ferrule 628 with shuttle 720. As shown in FIG. 7, the distal end 624 of cartridge 600 is inserted into aperture 703 at proximal end 706 of cartridge holder 700 until distal end 624 enters shuttle 720. Cartridge 600 continues to be pushed toward the distal end 704 of cartridge holder 700, until coding feature 610 on ferrule 628 mates with the coding feature 724 on shuttle 720. If the proper cartridge 600 is inserted, then the coding feature 610 will mate with coding feature 724, allowing the cartridge head to continue its trajectory and to be fully inserted into the shuttle 720. A properly mated ferrule 628 and shuttle 720 are shown in FIG. 8, where the cartridge head has been fully inserted into shuttle 720, thereby displacing clip 710 radially outwards from the shuttle 720.

FIG. 9 illustrates the fully inserted cartridge 600 within cartridge holder 700. As can be seen in FIG. 9, the ferrule 628 successfully mated with shuttle 720 and thus both shuttle 720 and ferrule 628 were able to be pushed all the way through the aperture 703 at distal end 704 of cartridge holder 700. The needle thread 722 on the exterior surface of shuttle 720 allows a needle to be affixed to the shuttle 720.

If an incorrectly coded cartridge 600 or a standard 3 ml cartridge 600 is inserted, the coding features within the shuttle 720 will block the cartridge head from being fully inserted into the shuttle 720. Thus the cartridge travel will be restricted, and the cartridge 600 will not be able to continue to be inserted within cartridge holder 700, as the clip 710 will not be displaced radially outwards from the shuttle 720.

A second clip, a retention clip 726 (shown in FIGS. 7 and 8), may be present and may be part of the shuttle 720 itself. This clip 726 may be pushed outward by cartridge 600 as the cartridge 600 is inserted into shuttle 720, shown in FIG. 7 and may then snap back into its original, or rest position as the clip 726 is released from contact with the ferrule 628 and instead abuts the smaller-diametered neck of cartridge 600, shown in FIG. 8. If cartridge 600 is later attempted to be removed from cartridge holder 700, shuttle 720 will remain attached and will travel with cartridge 600 until shuttle 720 reaches an indent 730 within the inner surface of holder 700. When retention clip 726 hits indent 730, the clip will flex outward to fill the space defined by indent 730, releasing cartridge 600 from shuttle 720. Shuttle 720 has now returned to its initial position, ready to accept the next correct cartridge 600.

In an alternative configuration, shuttle 720 may comprise a greater width than the cartridge diameter, allowing for other forms of cartridge dedication to govern the ability of a cartridge 600 to disengage clip 726, such as caps, coded labels, or features on the cartridge 600 itself.

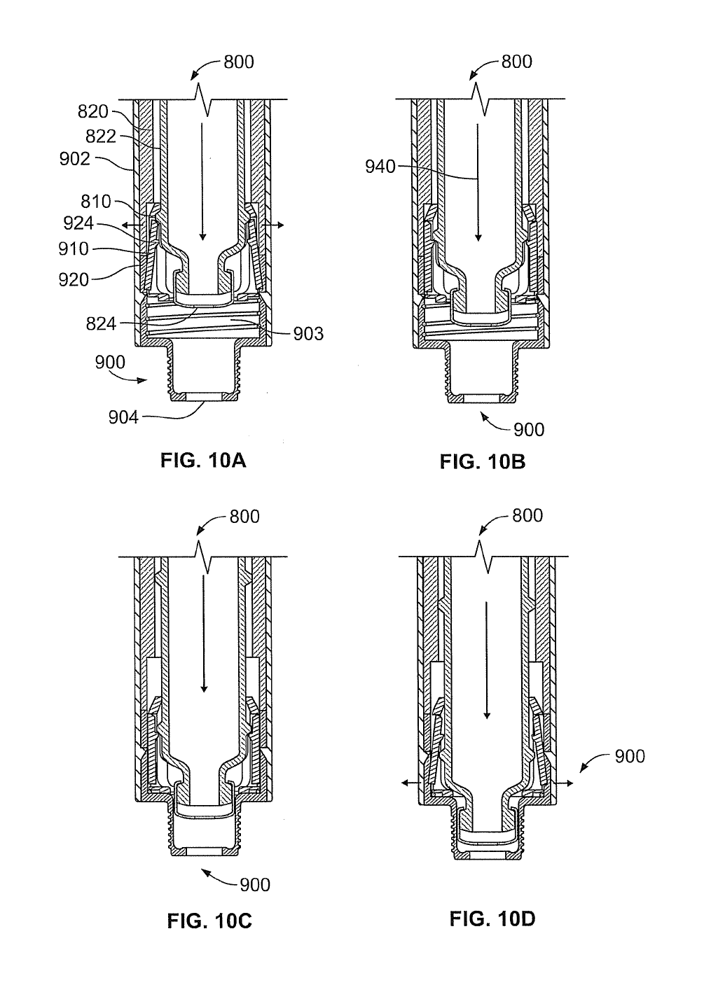

FIG. 10A illustrates a partial, cross-sectional view of yet another alternative arrangement of a cartridge 800 and cartridge holder 900. Cartridge 800 comprises a main body 820 with an exterior surface 822, a distal end 824, and a proximal end (not shown). Cartridge 800 is also fitted with at least one coding feature 810 on its exterior surface 822.

Cartridge holder 900 comprises a main body 902 defining an aperture 903, a distal end 904, and a proximal end (not shown). Cartridge holder 900 also comprises a shuttle 920 with at least one clip 910. Clip 910 may be deformable, with the ability to be pushed outward, toward the interior walls defining cartridge holder 900, when cartridge 800 is inserted. Shuttle 920 is preferably located toward the distal end 904 of cartridge holder 900, yet is not located at the distal end. The interior surface of shuttle 920 may comprise at least one coding feature 924.

In use, the distal end 824 of cartridge 800 is inserted into the proximal end of cartridge holder 900 via aperture 903. When cartridge 800 reaches shuttle 920, the coding features 810 serve to press against the interior surface of shuttle 920, pushing each clip 910 outward, and allowing cartridge 800 to continue to be inserted within shuttle 920. If cartridge 800 does not comprise a coding feature 810, or comprises an incorrect coding feature 810, the cartridge 800 will not be able to pass through shuttle 920 at this point, as clips 910 will not be pressed outward to allow the cartridge room to pass through.

FIG. 10B shows the cartridge 800 fully engaged within shuttle 920. As shown in FIG. 10B, the clips 910 on shuttle 920 are straight, and are parallel with the walls of cartridge 800 and cartridge holder 900. A coding feature 924 on the interior surface of shuttle 920, in this case a protrusion, serves to prevent coding feature 810 from proceeding further, which in turn prevents cartridge 800 from moving further down into shuttle 920. Thus, as cartridge 800 continues to be pushed axially toward distal end 904 of cartridge holder 900, coding feature 810 applies downward force on coding feature 924 of shuttle 920, which moves shuttle 920 toward distal end 904 of cartridge holder 900. The movement of shuttle 920 is shown by arrow 940.

FIG. 10C shows shuttle 920 after its trajectory, as it now abuts the surface of cartridge holder 900. Once in the position of FIG. 10C, cartridge 800 may continue to be pushed in the same direction, and the coding features 810 will now pass over coding features 924, allowing cartridge 800 to extend fully to the distal end, in the position of FIG. 10D.

The shuttle 920 may be designed to re-engage cartridge 800 on the proximal side of coding features 924. This way, if cartridge 800 is later pulled out and removed from cartridge holder 900, the shuttle 920 will move back up toward the holder proximal end along with the cartridge 800, so that the shuttle 920 can return to its initial position.

In alternative embodiments, shuttle 920 may have threads on the exterior surface for attaching needles, or may have additional locking means.

Those of skill in the art will recognize alternative geometries of these coding features 216, 416, 610, 724, 810, 924 may also be used. For example, the thickness or length of the coding mechanisms may be altered, and the gap may range in size as well. A change in any of these features may alter the force applied required to deform the coding mechanisms to affix them to the cartridge 120, 200, 400, 600, 800.

Although primarily aimed at the insulin market, the proposed coding schemes may apply to other drugs. In fact, the coding mechanism may apply to any drug delivery device 100, with any type of reservoir or primary pack, e.g. inhaler, pouch. Standard parts may be used on devices for dispensing all drugs, and the coding applied. One advantage of the proposed coding mechanism allows for a coding method to the cartridge holder 104, 300, 500, 700, 900 to prevent insertion of a cartridge 120, 200, 400, 600, 800 into the incorrect cartridge holder 104, 300, 500, 700, 900.

The proposed coding mechanism results in a number of advantages. The coding mechanism assists a user to distinguish between medicaments 125, thereby helping to ensure that a drug delivery device 100 can only be used with a medicament 125 for which the device is intended. Therefore, with the coding system applied to the cartridge 120, 200, 400, 600, 800, the cartridge 120, 200, 400, 600, 800 is prevented from being confused with any other drug by loading a cartridge 120, 200, 400, 600, 800 with an incorrect or unwanted interface. The coded system prevents a user from fully attaching the cartridge assembly onto an incorrect dose setting mechanism 102. In addition, with coding schemes, if a user attempts to load an incorrect cartridge assembly, the user will be alerted at an early stage of the assembly process, as the cartridge will 120, 200, 400, 600, 800 be blocked from entering the cartridge holder 104, 300, 500, 700, 900 at an early stage of insertion.

The coding mechanism also results in a low cost mechanism since the mechanisms do not require a large number of parts and can be manufactured in a cost effective manner. Moreover, because of the ease of alteration of the coding features 216, 416, 610, 724, 810, 924, there are quite a large number of different coding configurations between the cartridge holder 104, 300, 500, 700, 900 and cartridge 120, 200, 400, 600, 800 that may be used. Consequently, a large number of medicaments 125 can be distinguished from one another.

Exemplary embodiments have been described. However, as those of skill in the art will recognize certain changes or modifications to such arrangements may be made. As just one example, features discussed herein may be taken from one arrangement and combined with features of other arrangements. Those skilled in the art will understand, however, that changes and modifications may be made to these arrangements without departing from the true scope and spirit of the present invention, which is defined by the claims.

1-15. (canceled)

16. A cartridge assembly for use with a drug delivery device, the cartridge assembly comprising:
a cartridge holder defining an aperture therethrough, with a blocking feature and at least one holder coding feature;
a cartridge with at least one cartridge coding feature; and
a shuttle within the cartridge holder, wherein the shuttle comprises the blocking feature,
wherein when the cartridge is inserted into the cartridge holder, the cartridge coding feature mates with the holder coding feature, moving the blocking feature or allowing the blocking feature to move.

17. The cartridge assembly of claim 16 wherein the cartridge is inserted into the aperture at a proximal end of the cartridge holder.

18. The cartridge assembly according to claim 16 wherein the blocking feature is a blocking mechanism comprising the at least one holder coding feature, and wherein the blocking mechanism blocks at least a portion of an interior of the aperture; and wherein the blocking mechanism is moveable so that the cartridge can move through the cartridge holder.

19. The cartridge assembly of claim 18 wherein the blocking mechanism is a clip.

20. The cartridge assembly of claim 18 wherein the cartridge coding feature comprises at least one protrusion and/or at least one indentation.

21. The cartridge assembly of claim 16 wherein the cartridge is removable from the cartridge holder.

22. The cartridge assembly of claim 17, wherein the movement of the blocking feature allows for the cartridge to pass through the aperture.

23. The cartridge assembly of claim 16 wherein the holder coding feature is located on an interior surface of the shuttle.

24. The cartridge assembly of claim 16 wherein the cartridge coding feature is located on a ferrule at a distal end of the cartridge.

25. The cartridge assembly of claim 24 wherein when the ferrule is inserted within the shuttle, the holder coding feature mates with the cartridge coding feature, which releases the shuttle from its position within the cartridge holder, allowing for movement of the shuttle toward the distal end of the cartridge holder.

26. The cartridge assembly of claim 16 wherein the shuttle comprises at least one clip.

27. The cartridge assembly of claim 26 wherein the cartridge coding feature comprises a protrusion that pushes the clip away from the cartridge, allowing the cartridge to continue to pass through the shuttle.

28. A cartridge assembly for use with a drug delivery device, the cartridge assembly comprising:
a cartridge holder defining an aperture therethrough, with a blocking feature and at least one holder coding feature; and
a cartridge with at least one cartridge coding feature;
wherein when the cartridge is inserted into the cartridge holder, the cartridge coding feature mates with the holder coding feature, moving the blocking feature or allowing the blocking feature to move, and
wherein the blocking feature comprises a plurality of petals, that rotate inward to block at least a portion of the aperture, and rotate outward to remove the blockage of the portion of the aperture.

29. The cartridge assembly of claim 28 wherein the plurality of petals are attached to a rotating sleeve.

30. The cartridge assembly of claim 29 wherein the cartridge coding feature comprises a spherical protrusion that mates with a corresponding indent in the rotating sleeve, allowing rotation of the cartridge to translate to rotation of the rotating sleeve, which in turn translates to movement of the plurality of petals.
